(19) United States
(12) Patent Application Publication
(10) Pub. No.: US 2017/0044444 A1
(43) Pub. Date: Feb. 16, 2017

(54) PROCESS FOR SCAVENGING HYDROGEN SULFIDE PRESENT IN A FLUID STREAM

(71) Applicant: ECOLAB USA INC., St. Paul, MN (US)

(72) Inventors: Pramod Anandu SHANBHAG, Honnavar Uttara Kannada (IN); Hemant Surendra MONDKAR, Pune (IN); Rajganesh MEENAKSHI, AMbasamudram Taluk (IN)

(73) Assignee: ECOLAB USA INC., St. Paul, MN (US)

(21) Appl. No.: 15/118,588

(22) PCT Filed: Feb. 11, 2015
(86) PCT No.: PCT/US15/15461

§ 371 Date: Aug. 12, 2016

(30) Foreign Application Priority Data
Feb. 13, 2014 (IN) 668/CHE/2014

(51) Int. Cl. C10G 21/20; B01D 53/78; C07D 403/14; B01D 53/52

(52) U.S. Cl. CPC C10G 21/20; B01D 53/52; B01D 53/78; C07D 403/14; C10G 2300/207; C10G 2300/1051

(57) ABSTRACT

A process for scavenging hydrogen sulfide present in a fluid stream is provided. The process includes treating the fluid stream with a polytriazine, wherein the polytriazine converts the hydrogen sulfide to a corresponding thiol or thioether derivative, thereby reducing the amount of hydrogen sulfide present in the fluid stream.

### FIELD BACKGROUND

In general, the present disclosure relates to the field of hydrogen sulfide scavengers. More particularly, but not exclusively, the disclosure relates to polytriazines, processes for preparing polytriazines and use of polytriazines for scavenging hydrogen sulfide.

### DISCUSSION OF RELATED FIELD

Removal of sulphur based species from liquid or gaseous hydrocarbon streams is a challenge that is faced in several industries, including the oil and natural gas industries. The presence of sulphur based species, such as hydrogen sulfide, creates problems during various processes conducted in the oil industry, which include drilling, production, transportation, storage and processing of crude oil, and processing of waste water associated with the crude oil. The impact of hydrogen sulfide is not limited to the oil industry; it also creates problems in the natural gas industry. Hydrogen sulfide is a toxic gas that can result in acute and chronic health issues. Chemicals can be used to change the nature of the hydrogen sulfide making it a less volatile and less toxic compound to protect the personnel that work around streams containing hydrogen sulfide. Such chemicals are generally called scavengers.

In conventional techniques, triazine based scavengers have been widely used for removal of hydrogen sulfide from refinery streams. N-substituted s-triazine is one such example that has been widely used as a hydrogen sulfide scavenger. However, this conventional triazine based scavenger has a single s-triazine moiety to scavenge hydrogen sulfide. Hence, there are only two reactive sites in the molecule. Such scavengers are disclosed in, for example, U.S. patent application Ser. No. 12/723,434 and U.S. Pat. No. 7,264,786.

### SUMMARY

One embodiment of this disclosure provides a process for scavenging hydrogen sulfide present in a fluid stream. The process includes treating the fluid stream with polytriazine, wherein the polytriazine converts the hydrogen sulfide to corresponding thiol or thioether derivatives, thereby reducing the amount of hydrogen sulfide present in the fluid stream.

### BRIEF DESCRIPTION OF DRAWINGS

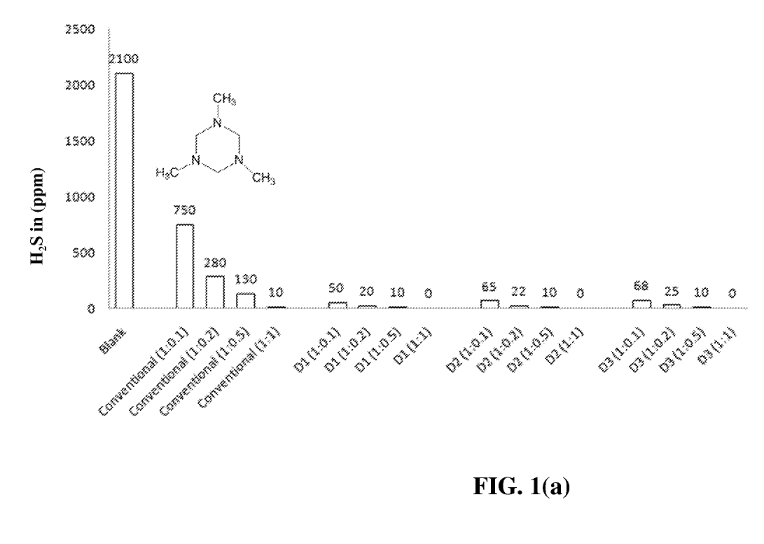

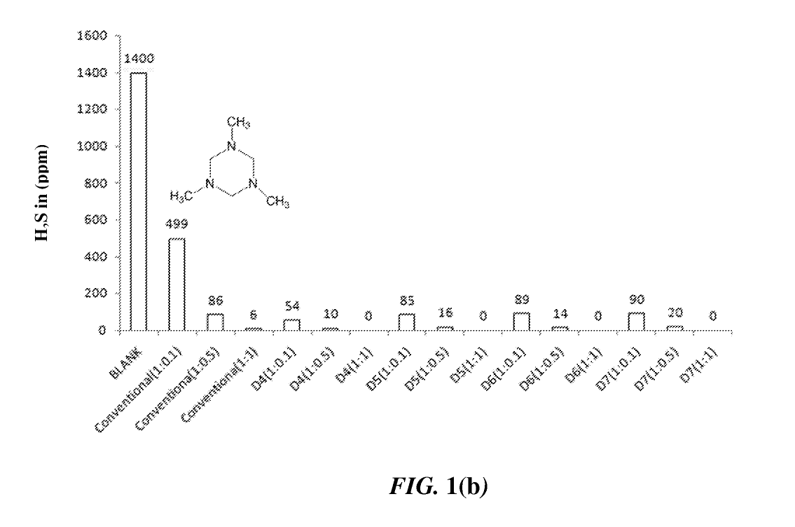

Embodiments are illustrated by way of example and not limitation in the Figures of the accompanying drawings, in which FIG. 1(a) and FIG. 1(b) are graphs illustrating a dosage profile of polytriazine derivatives and their efficacy to scavenge hydrogen sulfide in comparison with a conventional hydrogen sulfide scavenger.

### DETAILED DESCRIPTION

The following detailed description includes references to the accompanying drawings, which form part of the detailed description. The drawings show illustrations in accordance with exemplary embodiments. These exemplary embodiments are described in enough detail to enable those skilled in the art to practice the subject matter of the present disclosure. However, it will be apparent to one of ordinary skill in the art that the subject matter of the present disclosure may be practiced without certain specific details. In other instances, well-known methods and procedures have not been described in detail so as not to unnecessarily obscure aspects of the embodiments disclosed herein. The embodiments can be combined, other embodiments can be utilized, and structural and/or logical changes can be made without departing from the scope of the present disclosure. The following detailed description is, therefore, meant to be illustrative and non-limiting.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

An embodiment relates to the synthesis of polytriazine based compounds and their use as hydrogen sulfide and mercaptan scavengers. In some embodiments, the polytriazine based compound has the following formula (I):

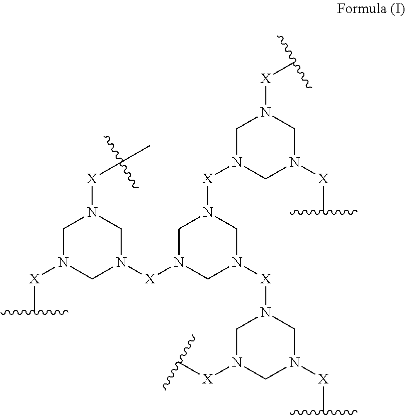

wherein “X” represents C1-C20 alkyl, C4-C7 cycloalkyl, C6-C18 aryl, C3-C7 alicyclic group or heterocyclic group with carbon ranging from C4-C6.

In an embodiment, X is an alkyl group. The alkyl group refers to straight or branched chain alkyl group, wherein the carbon chain ranges from C1 to C20, such as C1 to C10 or C1 to C6. The alkyl group may be, but is not limited to, methyl, ethyl, propyl, isopropyl, butyl, isobutyl, pentyl, isopentyl, neopentyl or hexyl.

In an embodiment, X is an aryl group with a carbon chain ranging from C6-C18. The aryl group used may be, for example, phenyl, napthyl or anthracyl. It shall be noted that other aryl groups can also be used.

In an embodiment, X is a cycloalkyl group, wherein the carbon chain ranges from C4to C7. The cycloalkyl group used may be, for example, cyclobutyl, cyclopentyl, cyclohexyl and/or cycloheptyl. The cycloalkyl may be substituted by lower alkyl groups or halogen atoms. The halogen atoms include fluorine, bromine, chlorine or iodine.

In an embodiment, X is a heterocyclic group selected from the group consisting of substituted or unsubstituted C4 to C6 heterocycles having 1-3 heteroatoms selected from N, O and S. Examples include, but are not limited to, pyrazine, pyrimidine, pyridiazine, furan, pyrrolodine and piperidine.

The heterocyclic group may be unsubstituted or substituted by halogen or lower alkyl groups. The halogen used can be, for example chlorine, iodine, fluorine, or bromine.

In a further embodiment, the heterocycles include carbon ring sizes ranging from C4 to C5.

In another embodiment, the six membered aromatic heterocycle having a C5 carbon ring size is pyridine. A person skilled in art may substitute other six membered heterocyclic groups to derive the compound of formula (I).

In another embodiment, the five membered aromatic heterocycle having a C4 carbon ring size is thiophene. It shall be noted that other five membered heterocyclic groups may also be used.

In an embodiment, X is an alicyclic group, wherein the carbon chain ranges from C3 to C7.

In an embodiment, polytriazines of formula (I) are synthesized by reacting a diamine with an aldehyde.

In an embodiment, the polytriazines are synthesized by reacting a diamine with aqueous formaldehyde under aqueous conditions.

In an embodiment, a 37% aqueous formaldehyde solution is used for reacting with the diamine. Further, other concentrations of aqueous formaldehyde, such as 30%-100%, may be used to react with the diamine for producing the compound of formula (I).

In some embodiments, the reaction temperature is maintained between about 40° C. and about 100° C.

In certain embodiments, the reaction temperature is maintained around 50° C.

In an embodiment, one mole of the diamine is added dropwise for a period of about 10 minutes to about 30 minutes to an aqueous formaldehyde solution, thereby forming a reaction mixture.

Further, in an embodiment, aqueous formaldehyde is added in excess amounts after completion of two hours of the reaction process to the aforementioned reaction mixture. On addition of excess aqueous formaldehyde, the reaction mixture is continuously stirred for about 6 hours to form a polymeric network.

In an embodiment, the excess aqueous formaldehyde added ranges from about 0.4 moles to about 20 moles.

In an embodiment, the time period to complete the reaction process is between about 8 and about 24 hours. The time period may vary based on the nature of diamine formed with respect to the spacers used.

In an embodiment, other forms of formaldehyde may also be used. Paraformaldehyde is one such example, among others.

Scheme (1) provided below discloses an exemplary synthesis of polytriazine using a diamine and formaldehyde.

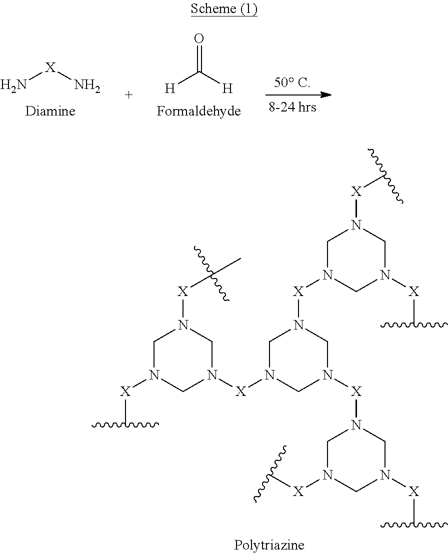

In an embodiment, the molar ratio of diamine to formaldehyde used in the reaction process ranges from about 1:2 to about 1:20.

In an embodiment, spacers in the diamine are selected from the group consisting of C1-C20 alkyl, C6-C18 aryl, C4-C7 cycloalkyl, C3-C7 alicyclic and C4-C8 heterocyclic spacers. The spacer separates the two amine groups in a diamine moiety (represented as X in the formula).

In an embodiment, the spacer used to prepare the diamine is a C1-C20 alkyl group. The alkyl group refers to straight or branched chain alkyl group, wherein the carbon chain ranges from C1 to C20, such as C1 to C10 or C1 to C6. The alkyl group may be, but is not limited to, methyl, ethyl, propyl, isopropyl, butyl, isobutyl, pentyl, isopentyl, neopentyl or hexyl.

Further, in an embodiment, the spacer used to prepare the diamine is selected from a C4-C7 cycloalkyl group. The cycloalkyl group used may be, but is not limited to, cyclobutyl, cyclopentyl, cyclohexyl and/or cycloheptyl. The cycloalkyl may be substituted by lower alkyl groups or halogen atoms.

In an embodiment, an aryl group is used as spacer to prepare the diamine. In some embodiments, the aryl groups are selected from C6-C18 aryl groups. In certain embodiments, the aryl group is selected from phenyl, napthyl and anthracyl. The phenyl, napthyl and anthracyl group can be optionally substituted with groups that do not react with formaldehyde during the reaction process. Moreover, it can be optionally substituted by halogen or members of lower alkyl groups.

“Optionally substituted” means that a group may or may not be further substituted or fused with one or more groups selected from hydrogen, lower alkyl groups, and halogen.

In an embodiment, the heterocyclic spacers used to prepare the diamine have carbon ring sizes ranging from C4-C7 having 1-3 heteroatoms selected from N, S or O. It shall be noted that the heterocyclic spacers used can be substituted or unsubstituted. Further, the substituted group may be one which does not react with formaldehyde during the reaction process.

In an embodiment, the heterocyclic compounds used may be optionally substituted by halogen or lower alkyl groups. Halogens include chlorine, fluorine, bromine and iodine.

In an embodiment, the heterocyclic compounds used to prepare the diamine are selected from the group consisting of six membered and five membered heterocyclic compounds.

In one embodiment, the six membered aromatic heterocyclic compound used to prepare the diamine is pyridine.

In another embodiment, the five membered aromatic heterocyclic compound used to prepare the diamine is thiopene.

A person skilled in art may also substitute other six or five membered heterocyclic compounds to derive the compound of formula (I).

Further, in an embodiment, the diamine used for the synthesis of the polytriazine is a symmetrical diamine.

In another embodiment, the diamine used for the synthesis of the polytriazine is an unsymmetrical diamine.

In an embodiment, polytriazine is used for scavenging hydrogen sulfide or mercaptans.

It shall be noted that a single molecule formed by the aforementioned process includes multiple s-triazine moieties. An increase in the number of s-triazine moieties provides more reactive sites in the polytriazine molecule and hence scavenges multiple moles of hydrogen sulfide from the stream.

The increase in the number of reactive sites in the polytriazine molecules enhances the scavenging properties of the molecules.

In an embodiment, the polytriazine includes at least two s-triazine moieties per molecule.

The stream from which hydrogen sulfide is scavenged using the polytriazines can be, for example, a fluid fuel stream that comprises hydrogen sulfide. The fluid fuel stream, for example, can be hydrocarbon fluid. The hydrocarbon fluid can be a complex mixture. Examples of hydrocarbon fluids are crude oil, vacuum gas oil, asphalt, fuel oil, distillate fuel, gasoline, diesel fuel, vacuum tower bottoms and other fluids produced from crude oil. The hydrocarbon fluid can be a liquid or a gas.

In an embodiment, an effective amount of polytriazine is brought into contact with a stream that comprises hydrogen sulfide. Polytriazines react with hydrogen sulfide upon contact and convert hydrogen sulfide to the corresponding thiol or thioether derivatives, thereby scavenging hydrogen sulfide from the stream. Thiols or thioethers formed by this method are less toxic in nature and are less odorous as compared to hydrogen sulfide.

In an embodiment, the process to scavenge hydrogen sulfide from the stream is carried out at ambient temperature. However, the temperature can vary from about 0° C. to about 300° C.

In some embodiments, the residence time of the polytriazine in the stream is about 2 hours to about 24 hours.

The amount of polytriazine used for scavenging may vary based on the amount of hydrogen sulfide present in the stream being treated.

The molar ratio of hydrogen sulfide to polytriazine may range from about 1:10 to about 1:0.1.

In an embodiment, a formulation comprising polytriazine, polar protic or polar aprotic solvents and a promoter is used for scavenging hydrogen sulfide from the stream comprising hydrogen sulfide.

In an embodiment, polytriazine is added to the stream by dissolving polytriazine in a solvent, such as, but not limited to, alcohols, hydrocarbons, ethers, aromatics, amides, nitriles, sulfoxides, esters and aqueous systems, among others.

In an embodiment, the formulation to scavenge hydrogen sulfide comprises about 65% water, about 30% polytriazine and about 5% promoter. Further, the composition of the formulation may vary as per specific requirements and is not limited to the aforementioned composition.

The promoter can be, for example, benzyl dialkyl decyl ammonium chloride, benzyl dialkyl dodecyl ammonium chloride, benzyl dialkyl tetradecyl ammonium chloride, and any combination thereof. It shall be note that other suitable long chain amine oxides can be used as the promoter. Promoters may also include ethoxylated alcohols, propoxylated alcohols or combinations thereof.

In an embodiment, the polytriazine is added neat to the stream to scavenge hydrogen sulfide from the stream.

### EXAMPLE 1

The following example is provided to further illustrate embodiments of the disclosure and should not be construed to limit the scope of the disclosure.

In this example, seven derivatives of polytriazine are presented, and their efficacy to scavenge hydrogen sulfide is illustrated.

### Derivative-1

Derivative-1 (D1) was prepared by adding ethylene diamine (12 grams, 0.2 mol) to a 37% aqueous solution of formaldehyde (17 grams, 0.2 mol) maintaining the temperature at 50° C. Ethylene diamine was added drop-wise to aqueous formaldehyde for a period of 30 minutes. Aqueous formaldehyde was added in excess amount (170 grams, 2 moles) after completion of two hours of the reaction process. Further, upon addition of excess aqueous formaldehyde, the reaction mixture was continuously stirred for additional 6 hours maintaining the temperature at 50°C. The completion of the reaction was monitored by thin layer chromatography (TLC) to confirm that no diamine was left to react. The product yield obtained was 82%. Scheme 2 provided below is the reaction scheme of synthesizing the instant polytriazine derivative, 1,3,5-tris(2-(3,5-dimethyl-1,3,5-triazinan-1-yl)ethyl)-1,3,5-triazinane.

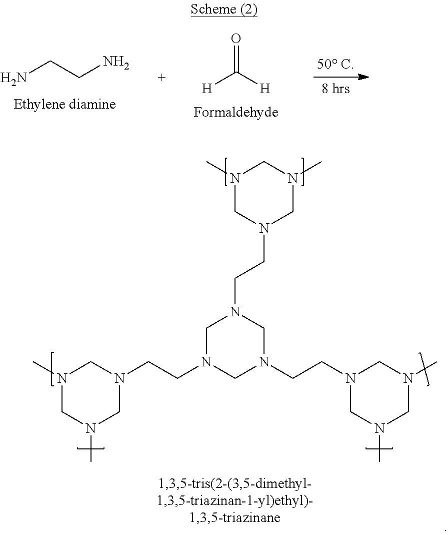

### Derivative-2

Derivative-2 (D2) was prepared by adding 1,3-diaminopropane (14.8 grams, 0.2mol) to a 37% aqueous solution of formaldehyde (17 grams, 0.2 mol) maintaining the temperature at 50° C. 1,3-diaminopropane was added drop-wise to aqueous formaldehyde for a period of 30 minutes. Aqueous formaldehyde was added in excess amount (170 grams, 2 moles) after completion of two hours of the reaction process. Further upon addition of excess aqueous formaldehyde, the reaction mixture was continuously stirred for additional 6 hours maintaining the temperature at 50°C. The completion of the reaction was monitored by thin layer chromatography (TLC) to confirm that no diamine was left to react. The product yield obtained was 85%. Scheme 3 provided below is the reaction scheme of synthesizing the instant polytriazine derivative, 1,3,5-tris(3-(3,5-dimethyl-1,3,5-triazinan-1-yl)propyl)-1,3,5-triazinane.

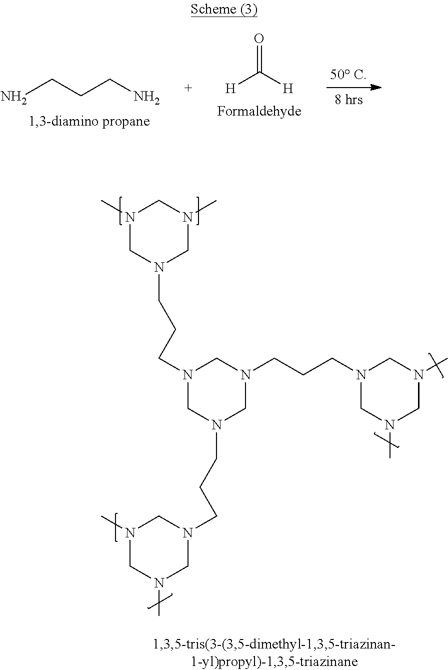

### Derivative-3

Derivative-3 (D3) was prepared by adding p-phenylenediamine (21.60 gram0.2 mol) to a 37% aqueous solution of formaldehyde (17 grams, 0.2 mol) maintaining the temperature at 50°C. p-phenylenediamine was added drop-wise to aqueous formaldehyde for a period 30 minutes. Aqueous formaldehyde was added in excess amount (170 grams, 2 moles) after completion of two hours of the reaction process. Further, upon addition of excess aqueous formaldehyde, the reaction mixture was continuously stirred for additional 6 hours maintaining the temperature at 50°C. The completion of the reaction was monitored by thin layer chromatography (TLC) to confirm that no diamine was left to react. The product yield obtained was 88%. Scheme 4 provided below is the reaction scheme of synthesizing the instant polytriazine derivative, 1,3,5-tris(4-(3,5-dimethyl-1,3,5-triazinan-1-yl)phenyl)-1,3,5-triazinane.

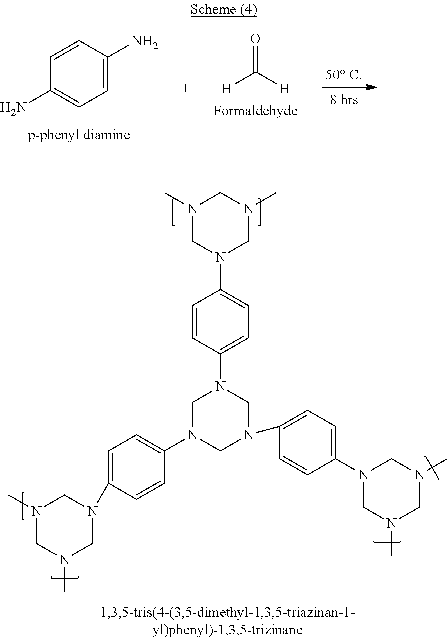

### Derivative-4

Derivative-4 (D4) was prepared by adding 1,4-diaminocyclohexane (22.8 grams, 0.2 mol) to a 37% aqueous solution of formaldehyde (17 grams, 0.2 mol) maintaining the temperature at 50°C. maintaining the temperature at 50° C. 1,4-diaminocyclohexane was added drop-wise to aqueous formaldehyde for a period of 30 minutes. Aqueous formaldehyde was added in excess amount (170 grams, 2 moles) after completion of two hours of the reaction process. Further upon addition of excess aqueous formaldehyde, the reaction mixture was continuously stirred for additional 22 hours maintaining the temperature at 50° C. The completion of the reaction was monitored by thin layer chromatography (TLC) to confirm that no diamine was left to react. The product yield obtained was 83%. Scheme 4 provided below is the reaction scheme of synthesizing the instant polytriazine derivative,1,3,5-tris(4-(3,5-dimethyl-1,3,5-triazinan-1-yl)cyclohexyl)-1,3,5-triazinane.

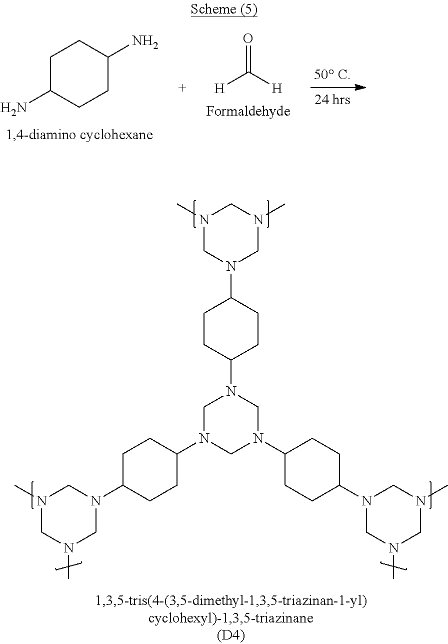

### Derivative-5

Derivative-5 was prepared by adding 1,5-diaminonaphthalene (31.6 grams, 0.2 mol) to a 37% aqueous solution of formaldehyde (17 grams, 0.2 mol) maintaining the temperature at 50°C. 1,5-diaminonaphthalene was added drop-wise to aqueous formaldehyde for a period of 30 minutes. Aqueous formaldehyde was added in excess amount (170 grams, 2 moles) after completion of two hours of the reaction process. Further upon addition of excess aqueous formaldehyde, the reaction mixture was continuously stirred for additional 22 hours maintaining the temperature at 50°C. The completion of the reaction was monitored by thin layer chromatography (TLC) to confirm that no diamine was left to react. The product yield obtained was 86%. Scheme 6 provided below is the reaction scheme of synthesizing the instant polytriazine derivative 1,3,5-tris(5-(3,5-dimethyl-1,3,5-triazinan-1-yl)naphthalen-1-yl)-1,3,5-triazinane.

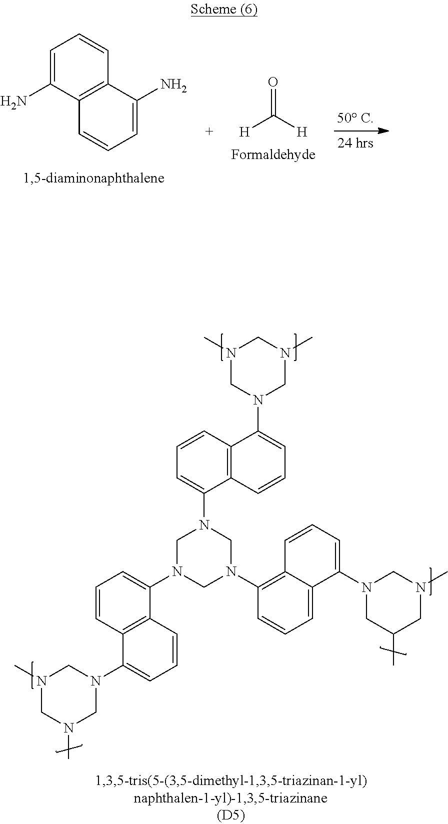

### Derivative-6

Derivative-6 was prepared by adding 2,6-diaminopyridine (21.8 grams, 0.2 mol) to a 37% aqueous solution of formaldehyde (17 grams, 0.2 mol) maintaining the temperature at 50°C. 2,6-diaminopyridine was added drop-wise to aqueous formaldehyde for a period of 30 minutes. Aqueous formaldehyde was added in excess amount (170 grams, 2 moles) after completion of two hours of the reaction process. Further upon addition of excess aqueous formaldehyde, the reaction mixture was continuously stirred for additional 22 hours maintaining the temperature at 50°C. The completion of the reaction was monitored by thin layer chromatography (TLC) to confirm that no diamine was left to react. The product yield obtained was 74%. Scheme 7 provided below is the reaction scheme of synthesizing the instant polytriazine derivative 1,3,5-tris(6-(3,5-dimethyl-1,3,5-triazinan-1-yl)pyridin-2-yl)-1,3,5-triazinane.

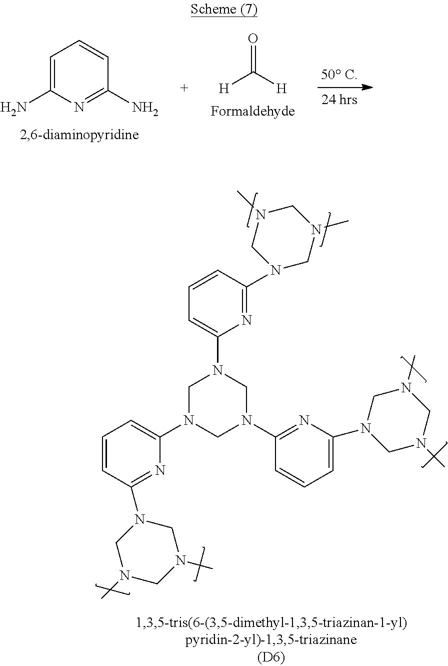

### Derivative-7

Derivative -7 was prepared by adding 3,4-diaminothiophene (22.8 grams, 0.2 mol) to a 37% aqueous solution of formaldehyde (17 grams, 0.2 mol) maintaining the temperature at 50° C. 3,4, diaminothiophene was added drop-wise to aqueous formaldehyde for a period of 30 minutes. Aqueous formaldehyde was added in excess amount (170 grams, 2 moles) after completion of two hours of the reaction process. Further upon addition of excess aqueous formaldehyde, the reaction mixture was continuously stirred for additional 22 hours maintaining the temperature at 50°C. The completion of the reaction was monitored by thin layer chromatography (TLC) to confirm that no diamine was left to react. The product yield obtained was 68%. Scheme 8 provided below is the reaction scheme of synthesizing the instant polytriazine derivative 1,3,5-tris(4-(3,5-dimethyl-1,3,5-triazinan-1-yl)thiophen-3-yl)-1,3,5-triazinane.

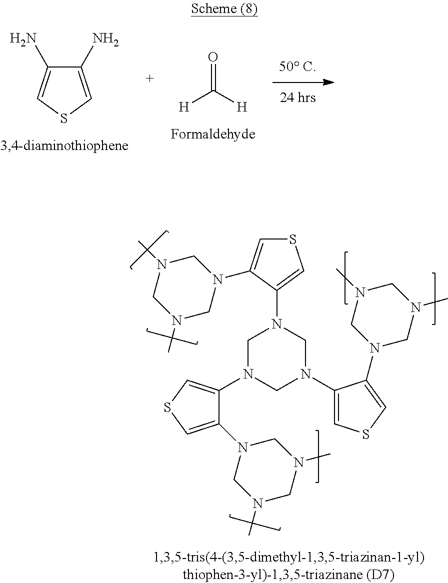

FIG. 1(a) and FIG. 1(b) are the graphs illustrating the dosage profile of the polytriazine derivatives and their efficacy to scavenge hydrogen sulfide in comparison with a conventional hydrogen sulfide scavenger. The efficacy of the polytriazine derivatives in scavenging hydrogen sulfide was evaluated by performing ASTM D-5705 Vapor phase test. In this graph, Y axis represents concentration of hydrogen sulfide in parts per million (ppm). A sample of kerosene treated with known amount of hydrogen sulfide was considered as blank. Further, similar samples were prepared and each sample was treated with different polytriazine derivatives and conventional N-alkyls-triazine scavenger to check their scavenging ability. It was observed that the derivatives had enhanced efficacy in scavenging hydrogen sulfide as compared to the conventional scavenger.

### CONCLUSION

The disclosed polytriazine derivatives have superior scavenging efficacy as compared to the conventional hydrogen sulfide scavengers.

A single molecule of a disclosed polytriazine derivative has multiple s-triazine moieties to scavenge hydrogen sulfide present in a stream.

Further, a single molecule of a disclosed polytriazine derivative has more reactive sites than conventional triazine based hydrogen sulfide scavengers. Hence, the disclosed polytriazine derivatives are more effective in scavenging hydrogen sulfide.

The processes described above is described as sequence of steps, which was done solely for the sake of illustration. Accordingly, it is contemplated that some steps may be added, some steps may be omitted, the order of the steps may be re-arranged or some steps may be performed simultaneously.

Although embodiments have been described with reference to specific exemplary embodiments, it will be evident that various modifications and changes may be made to these embodiments without departing from the broader spirit and scope of the compositions, systems and methods described herein. Accordingly, the specification and drawings are to be regarded in an illustrative rather than a restrictive sense.

Many alterations and modifications of the present disclosure may become apparent to a person of ordinary skill in the art after having read the foregoing description. It is to be understood that the phraseology or terminology employed herein is for the purpose of description and not of limitation. It is to be understood that the description above contains many specific examples. These should not be construed as limiting the scope of the disclosure but as merely providing illustrations of some exemplary embodiments of this disclosure. Thus, the scope of the disclosure should be determined by the appended claims and their legal equivalents rather than by the examples given.

1. A process for scavenging hydrogen sulfide present in a fluid stream,
wherein the process comprises, treating the fluid stream with a polytriazine,
wherein the polytriazine converts the hydrogen sulfide to a corresponding thiol or a thioether derivative, thereby reducing the amount of hydrogen sulfide present in the fluid stream.

2. The process according to claim 1, wherein the polytriazine has the formula: 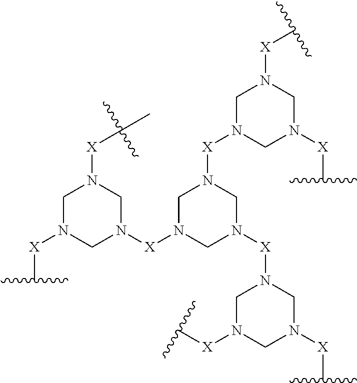
wherein X is selected from the group consisting of a straight or branched chain C1-C20 alkyl, C4-C7 cycloalkyl, C6-C18 aryl, C3-C7 alicyclic group and heterocycles with C4-C6 carbon ring size having 1-3 heteroatoms selected from N, O and S.

3. The process according to claim 1, wherein the fluid stream is a hydrocarbon fluid stream.

4. The process according to claim 1, further comprising preparing a formulation comprising the polytriazine, a solvent, and a promoter, and treating the fluid stream with the formulation.

5. The process according to claim 4, wherein the solvent is selected from the group consisting of a polar protic solvent, a polar aprotic solvent, water, and any combination thereof.

6. (canceled)

7. (canceled)

8. The process according to claim 1, wherein the polytriazine is added neat to the fluid stream.

9. (canceled)

10. The process according to claim 1, wherein a residence time provided for the polytriazine in the fluid is about 2 hours to about 24 hours.

11. The process according to claim 1, wherein a molar ratio of hydrogen sulfide to polytriazine is selected from a range between about 1:10 and about 1:0.1.

12. A compound having the formula: 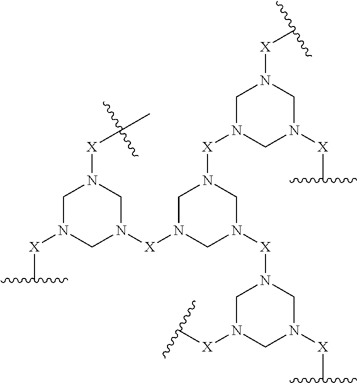
wherein X is selected from the group consisting of a straight or branched chain C1-C20 alkyl, C4-C7 cycloalkyl, C6-C18 aryl, C3-C7 alicyclic group and heterocycles with C4-C6 carbon ring size having 1-3 heteroatoms selected from N, O and S.

13. The compound according to claim 12, wherein the aryl group is selected from the group consisting of phenyl, napthyl and anthracyl.

14. The compound according to claim 12, wherein the six membered aromatic heterocyclic compound is pyridine and optionally the five membered aromatic heterocyclic compound is thiopine.

15. (canceled)

16. A process for preparing a polytriazine, the process comprising:
reacting formaldehyde with a diamine comprising a spacer, wherein the spacer is selected from the group consisting of a straight or branched chain C1-C20 alkyl, C4-C7 cycloalkyl, C6-C18 aryl, C3-C7 alicyclic and heterocycles with C4-C6 carbon ring sizes having 1-3 heteroatoms selected from N, O and S, wherein the diamine is added drop-wise to form a reaction mixture; and
adding formaldehyde to the reaction mixture.

17. The process according to claim 16, wherein the diamine is added drop-wise for a time period between about 15 minutes and about 30 minutes.

18. The process according to claim 16, wherein the aryl group is selected from the group consisting of phenyl, napthyl and anthracyl.

19. The process according to claim 16, wherein the six membered aromatic heterocyclic compound is pyridine and optionally the five membered aromatic heterocyclic compound is thiopine.

20. (canceled)

21. The process according to claim 16, wherein the cycloalkyl group is cyclohexyl.

22. The process according to claim 16, wherein a molar ratio of the diamine and the formaldehyde used in the reaction process ranges from about 1:2 to about 1:20.

23. The process according to claim 16, wherein the formaldehyde used is aqueous formaldehyde.

24. The process according to claim 16, wherein the diamine is reacted with the formaldehyde at a temperature ranging between about 40° C. and about 100°C.

25. The process according to claim 16, wherein the addition of formaldehyde to the reaction mixture ranges from about 0.4 moles to about 20 moles.
